(19) United States
(12) Patent Application Publication
(10) Pub. No.: US 2024/0296939 A1
(43) Pub. Date: Sep. 5, 2024

(54) PREDICTIVE MODEL FOR TESTING PRIOR TO VISIT

(71) Applicant: Cigna Intellectual Property, Inc., Wilmington, DE (US)

(72) Inventor: Rahib Diwan, Irvine, CA (US)

(21) Appl. No.: 18/115,835

(22) Filed: Mar. 1, 2023

(51) Int. Cl. G16H 40/20; G16H 10/40; G16H 50/70

(52) U.S. Cl. CPC G16H 40/20; G16H 10/40; G16H 50/70

(57) ABSTRACT

Methods and systems for recommending testing prior to a medical visit are provided. The methods and systems perform operations comprising: receiving a request to schedule an appointment with a medical professional; accessing patient information associated with a patient; and in response to receiving the request to schedule the appointment, applying a model to the patient information to conditionally trigger a recommendation to obtain one or more medical tests prior to the appointment.

### BACKGROUND

Patients seek medical treatment in a variety of different ways. Some patients desire medical treatment in person by visiting a live medical professional. Other patients prefer to receive medical treatment virtually. Different systems are provided to access medical treatments based on the preferences of the patients.

### BRIEF DESCRIPTION OF THE DRAWINGS

FIG. 1 is a block diagram of an example appointment scheduling platform, according to some embodiments.

FIG. 2 is an example database that may be deployed within the system of FIG. 1, according to some embodiments.

FIG. 3 is a block diagram of an example medical testing recommendation system that may be deployed within the system of FIG. 1, according to some embodiments.

FIGS. 4 and 5 are example user interfaces of the appointment scheduling platform, according to example embodiments.

FIG. 6 is a flowchart illustrating example operations of the appointment scheduling platform, according to example embodiments.

FIG. 7 is a block diagram illustrating an example software architecture, which may be used in conjunction with various hardware architectures herein described.

FIG. 8 is a block diagram illustrating components of a machine, according to some example embodiments.

FIG. 9 is a functional block diagram of an example neural network that can be used for the inference engine or other functions (e.g., engines) as described herein to produce a predictive model.

### DETAILED DESCRIPTION

Example methods and systems for platform with an appointment predictive scheduling module are provided. Specifically, the methods and systems provide recommendations for performing or obtaining one or more medical tests or examinations prior to a deadline, e.g., a scheduled appointment. In the following description, for purposes of explanation, numerous specific details are set forth in order to provide a thorough understanding of example embodiments. It will be evident, however, to one of ordinary skill in the art that embodiments of the invention may be practiced without these specific details.

Patients seek medical treatment in a variety of different ways. Some patients desire medical treatment in person by visiting a live medical professional. Other patients prefer to receive medical treatment virtually. Different systems are provided to access medical treatments based on the preferences of the patients. In order to access treatment of a particular type, a patient typically needs to launch a specific application associated with the desired treatment. The patient needs to navigate many pages of information to input their medical information, login and preferences to schedule and/or receive medical treatment of the particular type. If the patient, at a later time, seeks other types of service of care or medical treatment, a separate different application may need to be launched and accessed. Again, information needs to be reinput to receive and/or schedule treatment associated with the other type of service of care.

After a patient visits a health care professional, the health care professional may recommend that the patient obtain one or medical tests (e.g., bloodwork, imaging, urine tests, biopsies, or the like) from another facility or clinic. The patient may need to access another portal or application to schedule the testing that is recommended. Then, the patient needs to schedule another appointment with the health care professional to review the results of the medical tests. The patient again needs to navigate through the scheduling system and re-input various information to visit the health care professional. This causes the patient to not only waste a great deal of time scheduling the appointment and waiting in a medical office to see the health care professional but also to spend extra resources (e.g., money) on additional visits.

Typical systems fail to provide a single application that aggregates all of the different types of scheduling (e.g., testing and visiting a health care professional) into a single user interface and data record. In particular, providing a predictive model to have the testing complete and in the patient's data record prior to the visit with the care provider. Also, the typical systems fail to recognize the need to obtain tests for a medical professional to review before the appointment which wastes a great deal of time and resources. As a result, patients are burdened with having to navigate multiple pages of information and repeatedly inputting their information to receive treatment and patients are further burdened with repeated visits to the health care professional in relation to a same condition. The patient may also be burdened with insuring that the testing data is available in the provider record system prior to a visit. This is highly inefficient and wastes a great deal of time and resources that can be devoted to other tasks.

The disclosed embodiments provide systems and methods to recommend one or more medical tests or examinations to obtain prior to a scheduled appointment. Particularly, through the same interface used to schedule a visit to a health care professional, the disclosed embodiments access a patient profile that includes prior medical claims and electronic health records of the patient. Using the patient profile (and optionally a reason for the visit), a model, such as a machine learning model, estimates the need to obtain one or more medical tests prior to the scheduled visit. Through the same interface, the disclosed embodiments provide the ability for the patient to set up the appointment and identify medical tests that may be needed. The disclosed embodiments automatically generate a prescription record for the identified medical tests so that the patient can obtain the medical tests prior to the provider visit. In this way, results of the medical tests that may be needed for the visit are stored in the available health care (medical) professional database and to the health care (medical) professional at the time of the visit which reduces the need to schedule multiple visits for the same condition or reason.

As a result, a great deal of time and resources are saved and the user need not have to navigate through a multitude of pages of information to seek medical treatment or repeated input of their medical information into one or more medical databases. This saves time and reduces the amount of resources needed to accomplish a task.

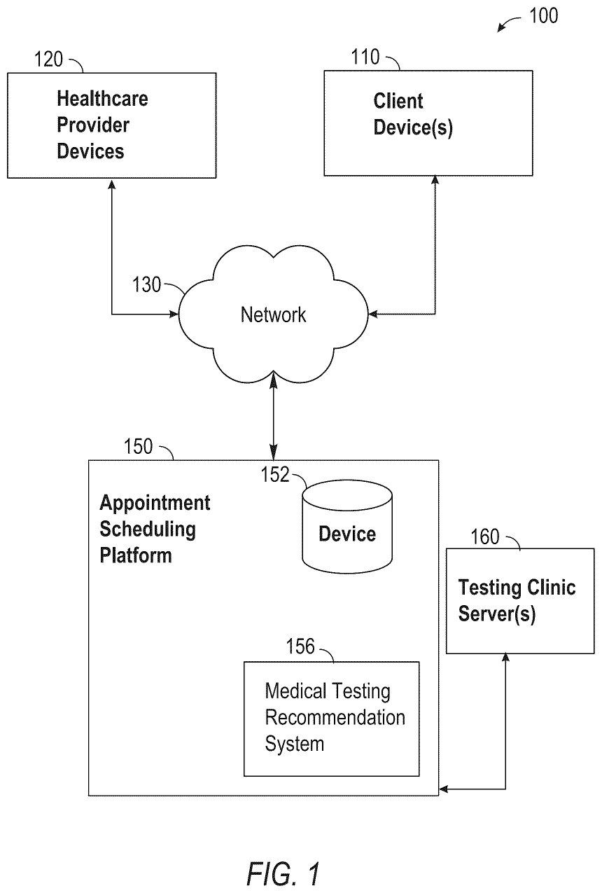

FIG. 1 is a block diagram showing an example appointment scheduling system 100 according to various exemplary embodiments. The appointment scheduling system 100 includes one or more client devices 110, one or more healthcare provider devices 120, an appointment scheduling platform 150, and one or more testing clinic servers 160 that are communicatively coupled over a communication network 130 (e.g., intranet, global computer network, Internet, telephony network or the like). Each of the one or more testing clinic servers 160 hosts an application that enables scheduling a medical testing at a particular center, clinic or facility.

The one or more testing clinic servers 160 can communicate with the appointment scheduling platform 150 to automatically receive a prescription and schedule a medical testing appointment at a geographically suitable location for a given patient. For example, a prescription record is generated and communicated to the medical testing database. In some cases, the patient, using an electronic device, interacts directly with the one or more testing clinic servers 160 to schedule an appointment for medical testing. In some cases, the patient interacts with the one or more testing clinic servers 160 indirectly through an interface of the appointment scheduling platform 150.

As used herein, the term “client device” may refer to any machine that interfaces to a communications network (such as network 130) to access the appointment scheduling platform 150. The client device 110 may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network or the appointment scheduling platform 150. The client device 110, when loaded with software associated with the present disclosure, is a dedicated machine.

In some cases, the appointment scheduling platform 150 is accessible over a global communication system, e.g., the Internet or world wide web. In such instances, the appointment scheduling platform 150 hosts a website (graphical user interface(s) and software to present data and receive input data) that is accessible to the client devices 110. Upon accessing the website, the client devices 110 provide secure login credentials, which are used to access a profile associated with the login credentials and one or more patient profiles or patient information. As used herein, patient information includes any medical information associated with a patient including one or more prior medical insurance claims that were approved or denied, one or more electronic health records or medical health records, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, and/or one or more treatment preferences. One or more user interfaces associated with the appointment scheduling platform 150 are provided over the Internet via the website to the client devices 110.

Healthcare provider devices 120 can include the same or similar functionality as client devices 110 for accessing the appointment scheduling platform 150. In some cases, the healthcare provider devices 120 are used by “internal” users. Internal users are medical professionals, such as medical personnel, physicians, clinicians, healthcare providers, health-related coaches pharmacy benefit manager (PBM) operators, pharmacists, specialty pharmacy operators or pharmacists, or the like that are associated with, certified by, or employed by one or more organizations that provides the appointment scheduling platform 150. In some cases, the healthcare provider devices 120 are used by “external” users. External users are medical professionals and personnel, such as physicians, clinicians, and health-related coaches that are associated with or employed by a different (external) organization than that which provides the appointment scheduling platform 150.

The healthcare provider devices 120 in some cases are used to access a respective server of the testing clinic servers 160. For example, a first group of healthcare provider devices 120 can access a first set of the testing clinic servers 160 that provide medical testing of a first type or that are affiliated with a first provider or organization. The first group and/or a second group of healthcare provider devices 120 can access a second set of the testing clinic servers 160 that provide medical testing of a second type or that are affiliated with a second provider or organization.

The healthcare provider devices 120, when used by internal or external users, to access the appointment scheduling platform 150 can view many records associated with many different patients (or users associated with client devices 110). Different levels of authorization can be associated with different internal and different external users to control which records the internal and external users have access. In some instances, only records associated with those patients to which a given internal or external user is referred, are made accessible and available to the given internal or external user device. Sometimes, a first internal or external user can refer a patient or records associated with the patient to a second internal or external user. In such circumstances, the second internal or external user becomes automatically authorized to access and view the patient's records that were referred by the first internal or external user.

The healthcare provider devices 120 can access healthcare scheduling platform 150 in order to review various recommendations that are provided to one or more patients by the medical testing recommendation system 156 prior to the patient arriving at an appointment. Specifically, the medical testing recommendation system 156 can generate an alert to the healthcare provider devices 120 that identifies a set of medical testing recommendations that were generated for a given patient. The alert can identify the patient, include various patient profile information that cause the medical testing recommendations to be generated and can identify the list of medical tests that were prescribed automatically or recommended by the medical testing recommendation system 156. The medical professional can review the information in the alert using the healthcare provider devices 120 to approve, modify, recall, or deny the automatically generated prescription. In some cases, the alert is only provided for a subset of patients that are randomly selected or for a subset of medical tests that are automatically recommended.

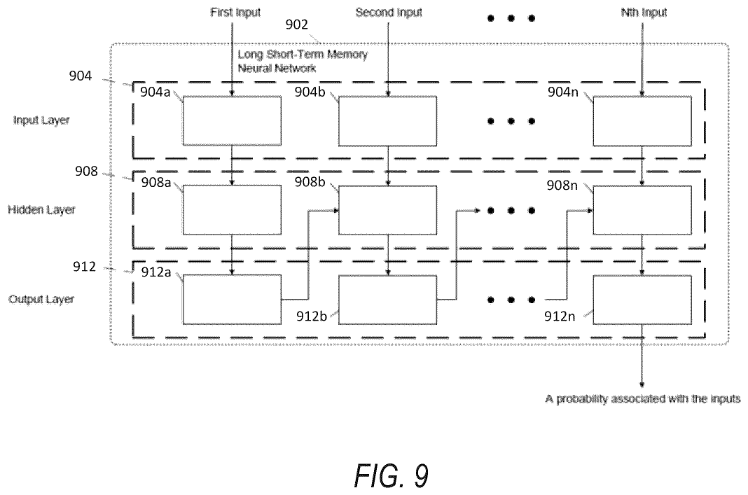

In some examples, the appointment scheduling platform 150 (and specifically the medical testing recommendation system 156) can implement a machine learning technique or machine learning model, such as a neural network (discussed below in connection with FIG. 9). The machine learning model can be trained to establish a relationship between a plurality of training patient information features and types of medical tests performed based on visits to various types of medical professionals. The machine learning model can then receive a new set of patient information for a patient scheduling a future medical visit and can estimate or predict one or more medical tests that the patient may need to obtain prior to arriving at the future medical visit. The machine learning model can prescribe automatically the one or more medical tests by communicating with the testing clinic servers 160. This allows the patient to schedule an appointment for a medical visit with a provider, order medical tests, and complete medical tests to be performed prior to the visit. In this way, the medical visit can be handled more efficiently by the medical professional as the necessary medical test results are available during the medical visit. This reduces the number of times the patient needs to visit the medical professional, improves the overall medical visit process, and reduces the number of pages of information and interfaces the patient has to navigate through to schedule a medical visit and associated medical tests. This may also reduce the data being created and stored in the medical system and the patient's electronic devices.

In an example, the machine learning model can be trained by obtaining a batch of training data comprising a first set of the plurality of training patient information features associated with a first type of medical test performed based on visits to a first type of medical professional. The machine learning model processes the first set of the plurality of training patient information features by the machine learning model to generate an estimated set of medical tests to obtain prior to a given appointment for the first type of medical professional. The machine learning model computes a loss based on a deviation between the estimated set of medical tests and the first type of medical test associated with the first set of the plurality of training patient information features and updates one or more parameters of the machine learning model based on the computed loss.

In some examples, the machine learning model is further trained by obtaining a second batch of training data comprising a second set of the plurality of training patient information features associated with a second type of medical test performed based on visits to a second type of medical professional. The machine learning model processes the second set of the plurality of training patient information features by the machine learning model to generate a second estimated set of medical tests to obtain prior to another given appointment for the second type of medical professional. The machine learning model computes a second loss based on a deviation between the second estimated set of medical tests and the second type of medical test associated with the second set of the plurality of training patient information features and updates the one or more parameters of the machine learning model based on the computed second loss.

In some examples, the machine learning model is further trained by obtaining a third batch of training data comprising a third set of the plurality of training patient information features associated with a third type of medical test performed based on visits to the second type of medical professional. The machine learning model processes the third set of the plurality of training patient information features by the machine learning model to generate a third estimated set of medical tests to obtain prior to the another given appointment for the second type of medical professional. The machine learning model computes a third loss based on a deviation between the third estimated set of medical tests and the third type of medical test associated with the second set of the plurality of training patient information features and updates the one or more parameters of the machine learning model based on the computed third loss.

These training operations can be repeated for multiple batches of training data and/or until a stopping criterion is reached.

In some examples, the medical testing recommendation system 156 receives a request to schedule an appointment with a medical professional. The medical testing recommendation system 156 accesses patient information associated with a patient. The medical testing recommendation system 156, in response to receiving the request to schedule the appointment, applies a model (e.g., the machine learning model) to the patient information to conditionally trigger a recommendation to obtain one or more medical tests prior to the appointment.

In some examples, the medical testing recommendation system 156 identifies, by the model, the one or more medical tests based on the patient information. In some aspects, the medical testing recommendation system 156 determines, by the model, that the one or more medical tests are needed prior to the appointment. The medical testing recommendation system 156, in response to determining by the model that the one or more medical tests are needed prior to the appointment, automatically generates a prescription to perform the one or more medical tests and sends the prescription to a facility (e.g., testing clinic servers 160) associated with the patient to conduct the one or more medical tests.

In some examples, the medical professional or provider includes a primary care physician (PCP) and the one or more medical tests include at least bloodwork. In some aspects, the appointment includes an annual checkup. In such cases, the medical testing recommendation system 156 selects the one or more medical tests from a plurality of available tests based on an output of the model.

In some examples, the patient information includes at least one information for the patient, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, or one or more treatment preferences. In some examples, the medical testing recommendation system 156 applies the model by: generating a profile of patients associated with the one or more medical tests and determining that one or more attributes of the patient information match the profile of the patients. The model can be applied in response to determining that the one or more attributes of the patient information match the profile of the patients.

In some examples, the medical testing recommendation system 156 determines based on the patient information a date associated with a last visit the patient had with any medical professional. The medical testing recommendation system 156 prevents recommending obtaining the one or more medical tests in response to determining that the date of the last visit is less than a threshold period of time of a current date. For example, if the patient recently visited another medical professional (e.g., in the past 90 days) or if the patient has already seen this same medical professional for which the appointment is being scheduled within the past year, the medical testing recommendation system 156 can determine that there is no need to perform medical tests prior to the scheduled visit. This may be because the medical tests may have already been performed as a result of the past visit.

In some cases, the medical testing recommendation system 156 can generate a set of medical tests that may be needed for the scheduled visit. The medical testing recommendation system 156 can search the patient profile to determine whether one or more medical tests of the set of medical tests were previously obtained and stored in the patient profile. For example, such medical tests may have been performed by another facility (not associated with the facility of the medical professional for which the visit is being scheduled) recently (e.g., within the past 6 months) and results of the medical tests may be stored in the patient profile that is accessible to both facilities. The medical testing recommendation system 156 can compare the date of the previously obtained and stored medical tests from the patient profile and determine whether the date that the medical tests were obtained is within a threshold date range or time interval (e.g., 6 months). In response to determining that the previously obtained and stored medical tests were obtained and stored on a date that is within the threshold date range or time interval, the medical testing recommendation system 156 avoids or prevents recommending the same tests to be obtained. In such cases, the medical testing recommendation system 156 removes the previously obtained and stored medical tests from the set of medical tests that were identified for the upcoming scheduled visit. This further improves the overall efficiency of the system and prevents unnecessary medical tests from being obtained and performed, for example in cases where a first medical facility may not be aware that medical tests were already performed by a second facility and that results of such tests can be used by the first facility. In such aspects, the model determines that the one or more tests are not needed prior to the appointment and prevents triggering a recommendation to perform the one or more tests prior to the appointment.

In some examples, the appointment is scheduled for a first date, the one or more medical tests are prescribed to be obtained at a second date that precedes the first date, and results of the one or more medical tests are made available to the medical professional prior to the first date of the appointment. In some aspects, the patient information includes results of prior medical tests and diagnosis performed by one or more clinics that are different from a clinic associated with the medical professional. In some examples, the request to schedule the appointment (e.g., an electronic record transmitted to an insurance database from the provider system) includes a reason for the appointment. In such cases, the recommendation to obtain the one or more medical tests prior to the appointment is further generated by the model being applied to the reason for the appointment.

In some examples, the medical testing recommendation system 156 receives feedback from the medical professional after the appointment with the patient. The feedback can be an electronic record. The feedback can indicate a level of importance of the one or more medical tests that were automatically recommended and obtained by the patient prior to the appointment. The medical testing recommendation system 156 can retrain the model by updating one or more parameters of the model based on the triggered recommendation and the level of importance indicated in the received feedback to refine future predictions made by the model.

The network 130 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, subsequent wireless network protocols, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The healthcare provider devices 120 can be used to access pharmacy claims, medical data (e.g., medical information 230 stored in database 152), laboratory data and the like for one or more patients that the healthcare provider devices 120 are authorized to view. This patient information 210 can be maintained in a database 152 by the appointment scheduling platform 150 or in a third-party database accessible to the appointment scheduling platform 150 and/or the healthcare provider devices 120.

In some embodiments, the client devices 110 and the appointment scheduling platform 150 can be communicatively coupled via an audio call (e.g., VOIP, Public Switched Telephone Network, cellular communication network, etc.) or via electronic messages (e.g., online chat, instant messaging, text messaging, email, and the like). While FIG. 1 illustrates a single client device 110 and a single healthcare provider device 120, it is understood that a plurality of such devices can be included in the system 100 in other embodiments. As used herein, the term “client device” may refer to any machine that interfaces to a communications network (such as network 130) to obtain resources from one or more server systems or other client devices.

In some embodiments, the appointment scheduling platform 150 includes the medical testing recommendation system 156. In some examples, the medical testing recommendation system 156 processes medical information input by a patient via the client device 110 and/or patient information stored in one or more databases. For example, the client device 110 can present a graphical user interface to the patient. The graphical user interface can receive input from the patient that provides a variety of medical information, such as patient health information, patient demographic information, patient in-network insurance coverage, patient out-of-network insurance coverage, patient location, and/or one or more treatment preferences. The input can also identify storage locations of one or more electronic health records and/or claims information.

In some cases, the patient health information can be input by receiving selection from the patient of one or more checkboxes from a user interface, each associated with a different medical condition, such as high blood pressure, diabetes, obesity, demographics, and so forth. The patient in-network insurance coverage and out-of-network insurance coverage be input by receiving selection from the patient of one or more checkboxes from a user interface, each associated with a different health insurance carrier. Based on the selected health insurance carrier and health plan information input by the patient, the patient in-network and out-of-network insurance coverages can be automatically determined and retrieved. The patient location can be automatically determined by accessing location information (e.g., GPS coordinates) from the client device 110 and/or by requesting a residential address from the patient. The treatment preferences can be input by receiving selection from the patient specifying how the patient likes to receive treatment, such as in-person, virtually, in a structured format, in aspirational format (e.g., a format that lacks structure), and so forth.

The medical testing recommendation system 156 can store a previously trained machine learning model. The machine learning model can process the information input by the patient (which can include a reason for the visit), a future date of the visit, and patient information. The medical testing recommendation system 156 can conditionally trigger a recommendation to obtain one or more medical tests based on the information input by the patient, the future date of the visit, and the patient information. If the medical testing recommendation system 156 triggers a recommendation to obtain one or more medical tests, the medical testing recommendation system 156 can communicate a notification to the client device 110 that identifies the one or more medical tests to obtain before the scheduled future date of the visit and/or can communicate with the testing clinic servers 160 a prescription for the one or more identified medical tests. Such a communication can be sending a machine readable packet representing the testing needed, as well as a date and time for the testing.

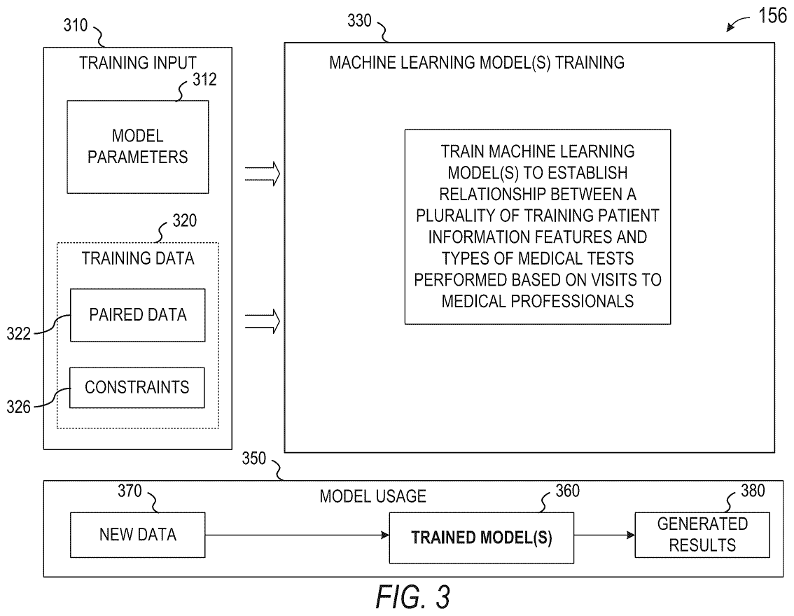

In some examples, as explained in more detail in connection with FIG. 3, the medical testing recommendation system 156 is trained based on training patient information features (e.g., electronic health record, past claims information for training patients, patient health information for training patients, patient demographic information for training patients, prior bloodwork results for training patients, prior results of non-bloodwork tests for training patients, medical history for training patients, medical provider notes in the electronic health record for training patients, intake forms completed by training patients, in-network insurance coverage for training patients, out-of-network insurance coverage for training patients, locations of training patients, or one or more treatment preferences for training patients) and their corresponding ground-truth medical tests performed or prescribed based on visits to medical professionals. Any of this data may be part of an electronic record. The medical testing recommendation system 156 is trained to predict one or more medical tests to recommend to have performed or be obtained before a scheduled appointment for a given set of patient information features. For example, the medical testing recommendation system 156 can analyze the training data to identify which types of medical tests were prescribed immediately after or during a medical visit for a given set of training patient information features. The prediction can be used to recommend or not recommend one or more medical tests to obtain prior to a scheduled visit. The prediction can be a machine readable record.

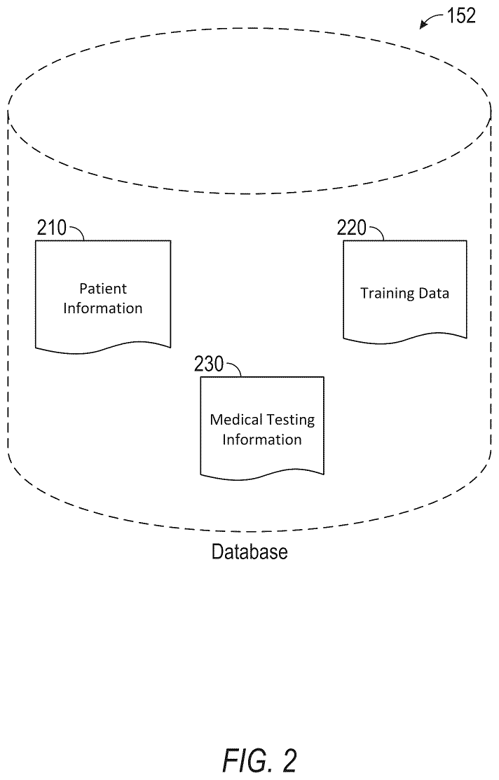

FIG. 2 is an example database 152 that may be deployed within the system of FIG. 1, according to some embodiments. The database 152 includes patient information 210, medical testing information 230, and training data 220. The patient information 210 can be generated or accessed by the appointment scheduling platform 150. For example, the appointment scheduling platform 150 can access one or more patient records from one or more sources, including pharmacy claims, benefit information, prescribing physician information, dispensing information (e.g., where and how the patient obtains their current medications), demographic information, prescription information including dose quantity and interval, and input from a patient received via a user interface presented on the client device 110 and so forth. The appointment scheduling platform 150 can collect this information from the patient records and generates a patient features vector that includes this information.

The medical testing information 230 stores various results of medical tests and examinations related information that were performed for each set of patients. The medical testing information 230 can also store an association between reasons for visits to a medical professional and types of medical tests performed or needed for such visits. The medical testing information 230 may also store the medical tests that are ordered by a type of provider, e.g., the practice group in which the provider practices, the historical medical tests order records by similar providers (a similar provider can be a pediatrician, adult primary care physician, a cardiologist, an orthopedist, or the like). The medical testing information 230 can store a feature vector that associates different patient features, reasons for visits, and types of medical professional associated with such visits and one or more medical tests performed or needed for such visits. The medical testing recommendation system 156 can access the medical testing information 230 and search the medical testing information 230 based on newly input patient information and a reason for a visit to identify the one or more medical tests to recommend to have performed before the scheduled visit. In some cases, the medical testing information 230 stores parameters of the machine learning model that generates the recommendations for the medical tests to have performed before a visit using the patient information (which can include a reason for a visit).

The training data 220 includes training sets of the plurality of training patient information features associated with types of medical tests performed based on visits to various types of medical professionals. The training data 220 is used to train a machine learning model implemented by the service of medical testing recommendation system 156 to generate estimates of one or more medical tests to recommend or not recommend to a patient to obtain prior to arriving at a scheduled visit. For example, the training data 220 can be built over time by identifying a first set of the plurality of training patient information features that are associated with a given set of medical tests performed in response to a particular visit to a particular medical professional and by identifying a second set of the plurality of training patient information features that are associated with a second given set of medical tests performed in response to another particular visit to another particular medical professional. The training data 220 can also be built around a large database of patient information, e.g., terabytes. The model also takes into account the past recommendations for testing based on patients similar to the present patient who has a visit to a medical provider.

FIG. 3 is a block diagram of an example service of medical testing recommendation system 156 that may be deployed within the system of FIG. 1, according to some embodiments. Training input 310 includes model parameters 312 and training data 320 (e.g., training data 220 (FIG. 2)) which may include paired training data sets 322 (e.g., input-output training pairs) and constraints 326. Model parameters 312 stores or provides the parameters or coefficients of corresponding ones of machine learning models. During training, these parameters 312 are adapted based on the input-output training pairs of the training data sets 322. After the parameters 312 are adapted (after training), the parameters are used by trained models 360 to implement the trained machine learning models on a new set of data 370.

Training data 320 includes constraints 326 which may define the constraints of a given patient information features. The paired training data sets 322 may include sets of input-output pairs, such as pairs of a plurality of training patient information features and types of medical tests performed based on visits to various types of medical professionals associated with the training patient information features. Some components of training input 310 may be stored separately at a different off-site facility or facilities than other components.

Machine learning model(s) training 330 trains one or more machine learning techniques based on the sets of input-output pairs of paired training data sets 322. For example, the model training 330 may train the machine learning (ML) model parameters 312 by minimizing a loss function based on one or more ground-truth type of service of care. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, the ML model can be applied to a training plurality of patient information features to estimate or generate a prediction of types of medical tests to have performed prior to a visit to a type of medical professional. The ML model may also take into account the known demographics and health information of the patient who has an appointment with the medical professional. In some implementations, a derivative of a loss function is computed based on a comparison of the estimated types of medical tests and the ground truth types of medical tests associated with the training patient information features and parameters of the ML model are updated based on the computed derivative of the loss function.

In one example, the ML model receives a batch of training data that includes a first set of the plurality of training patient information features associated with a ground truth first type of medical test performed based on visits to a first type of medical professional. The ML model generates a feature vector based on the first set of the plurality of training patient information features and generates an estimated set of medical tests to obtain prior to a given appointment for the first type of medical professional. The prediction is compared with the ground truth first type of medical test performed based on visits to the first type of medical professional and one or more parameters of the ML model are updated based on the comparison.

The result of minimizing the loss function for multiple sets of training data trains, adapts, or optimizes the model parameters 312 of the corresponding ML models. In this way, the ML model is trained to establish a relationship between a plurality of training patient information features and types of medical tests performed based on visits to various types of medical professionals.

For example, the ML model receives a second batch of training data comprising a second set of the plurality of training patient information features associated with a ground truth second type of medical test performed based on visits to a second type of medical professional. The ML model generates a feature vector based on the second set of the plurality of training patient information features and generates an estimated second estimated set of medical tests to obtain prior to another given appointment for the second type of medical professional. The prediction is compared with the ground truth second type of medical test performed based on visits to the second type of medical professional and one or more parameters of the ML model are updated based on the comparison.

As a further example, the ML model receives a third batch of training data comprising a third set of the plurality of training patient information features associated with a ground truth third type of medical test performed based on visits to the second type of medical professional. The ML model generates a feature vector based on the third set of the plurality of training patient information features and generates a third estimated set of medical tests to obtain prior to the another given appointment for the second type of medical professional. The prediction is compared with the ground truth third type of medical test performed based on visits to the second type of medical professional and one or more parameters of the ML model are updated based on the comparison.

After the machine learning model is trained, new data 370, including one or more patient information features are received. The trained machine learning technique may be applied to the new data 370 to generate results 380 including a prediction of a need to have one or more medical tests performed prior to a visit to a medical professional along with types of medical tests to have performed. The recommendation made by the medical testing recommendation system 156 can be represented in a graphical user interface that depicts each of a plurality of different types of medical testing to obtain.

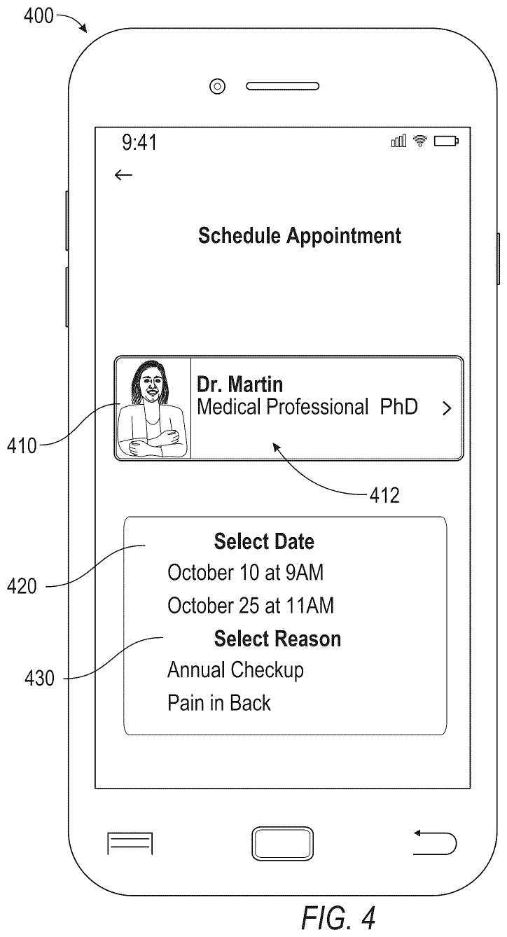

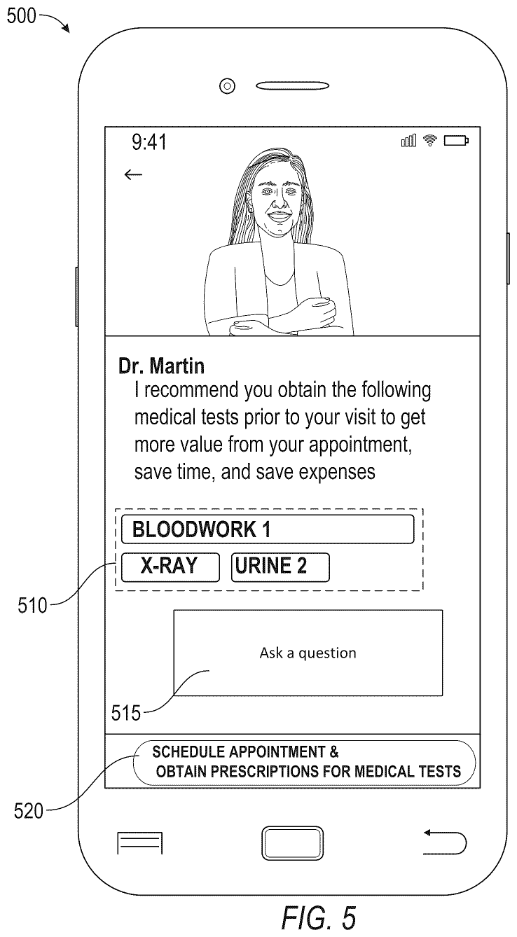

FIGS. 4 and 5 are example user interfaces 400 and 500 of the appointment scheduling system 100, according to example embodiments. For example, a client device 110 can provide medical information (patient information) associated with a patient to the medical testing recommendation system 156. To do so, the client device 110 can present a graphical user interface through which the patient inputs various patient information. The patient can also input a request for medical services, such as a request to schedule an appointment with a particular medical professional (e.g., a physician).

The user interface 400 can present an identifier of the medical professional 410 including a description of the type of service 412 provided by the medical professional 410. The user interface 400 can receive input from the client device 110 that selects the identifier of the medical professional 410. In response, the user interface 400 retrieves a list of available future dates on which a visit to the medical professional 410 are available. For example, the user interface 400 presents a date region 420 with options to select a particular future date and time from a list of future dates and times. The user interface 400 also presents a reason region 430. The reason region 430 includes a list of types of service of care associated with the medical professional 410. The 400 can receive input from the client device 110 that selects one or more reasons from the listed regions in the reason region 430.

The medical testing recommendation system 156 receives patient information associated with the patient scheduling the visit and receives the reason selected from the reason region 430. The medical testing recommendation system 156 applies the machine learning model to the patient information and/or the reasons and/or the type of service 412 provided by the medical professional 410 to estimate a need for one or more medical tests to be performed before the date and time selected from the date region 420. For example, the medical testing recommendation system 156 can determine or estimate and identify one or more medical tests that may be needed for the scheduled visit.

In response, the medical testing recommendation system 156 presents the user interface 500 to the patient on the client device 110. The user interface 500 includes a description of the type of service 412 provided by the medical professional 410. The user interface 500 also includes a list of identified medical tests 510 that are predicted and estimated by the medical testing recommendation system 156 based on the application of the model to the patient information and/or the reasons and/or the type of service 412. For example, the user interface 500 can recommend that the patient perform or obtain bloodwork of a certain type, urine tests, and an x-ray prior to the date and time selected from the date region 420. The user interface 500 can also include a region 515 where the patient can input questions that are routed to the medical professional 410 to allow the patient to chat briefly with the medical professional 410 ahead of the scheduled appointment. Once the patient is satisfied with the appointment date and time and receives the prompt or information with the identified medical tests 510 that are estimated by the medical testing recommendation system 156, the patient can select an option 520 to schedule the appointment and obtain prescriptions for the identified medical tests 510.

In response to receiving selection of the option 520, the prescriptions are routed to the one or more testing clinic servers 160 and a new interface is presented to the user to select a suitable date and time (that precedes the date and time of the appointment selected from the date region 420) for obtaining the identified medical tests 510. A confirmation prompt can be presented that identifies and confirms the appointment for the date and time selected from the date region 420 and that confirms the date and time for obtaining the identified medical tests 510 prior to the date and time of the appointment selected from the date region 420.

In some examples, the medical testing recommendation system 156 considers recommendations made by the medical professional 410 and/or other medical professionals that may be stored in the patient information in generating the identified medical tests 510. For example, if the patient has a history of iron deficiency and on prior visits the patient was recommended to take iron supplements, the medical testing recommendation system 156 can identify this recommendation and suggest as identified medical tests 510 a medical test for iron deficiency. This way, the medical professional 410 can analyze the results of the medical test to determine if the patient should continue taking the iron supplements on the date and time of the appointment selected from the date region 420.

The medical testing recommendation engine 156 can further act to schedule a patient appointment for testing with a medical facility computer. The medical facility can the testing or lab work to the patient. The medical facility computer can provide assign an appointment to a patient device upon the predictive model producing a predictive output that the patient will need scheduled lab work or other medical procedure prior to seeing a medical provider. A dynamic schedule component can automatically adjust the schedule based upon communication between the patient device and the medical facility computer.

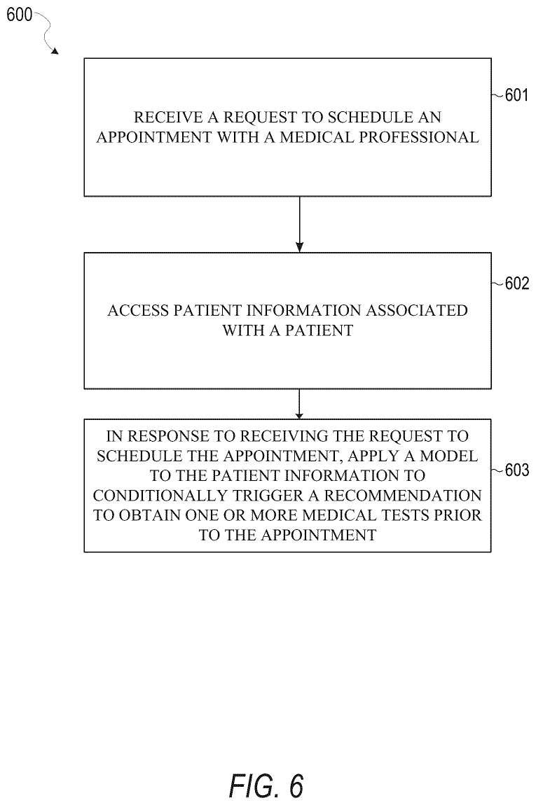

FIG. 6 is a flowchart illustrating example operations of the appointment scheduling system in performing a method or process 600, according to example embodiments. The process 600 may be embodied in computer-readable instructions for execution by one or more processors such that the operations of the process 600 may be performed in part or in whole by the functional components of the system 100; accordingly, the process 600 is described below by way of example with reference thereto. However, in other embodiments, at least some of the operations of the process 600 may be deployed on various other hardware configurations. Some or all of the operations of process 600 can be in parallel, out of order, or entirely omitted.

At operation 601, the system 100 receives a request to schedule an appointment with a medical professional, as discussed above. In an example, the reception of a request is, at least in part, an electronic communication.

At operation 602, the system 100 accesses patient information associated with the patient, as discussed above. In an example, accessing patient information can include accessing a record in a database and electronically communicating the patient information.

At operation 603, the system 100, in response to receiving the request to schedule the appointment, applies a model to the patient information to conditionally trigger a recommendation to obtain one or more medical tests prior to the appointment, as discussed above. The recommendation can be an electronic order or prescription for a medical test.

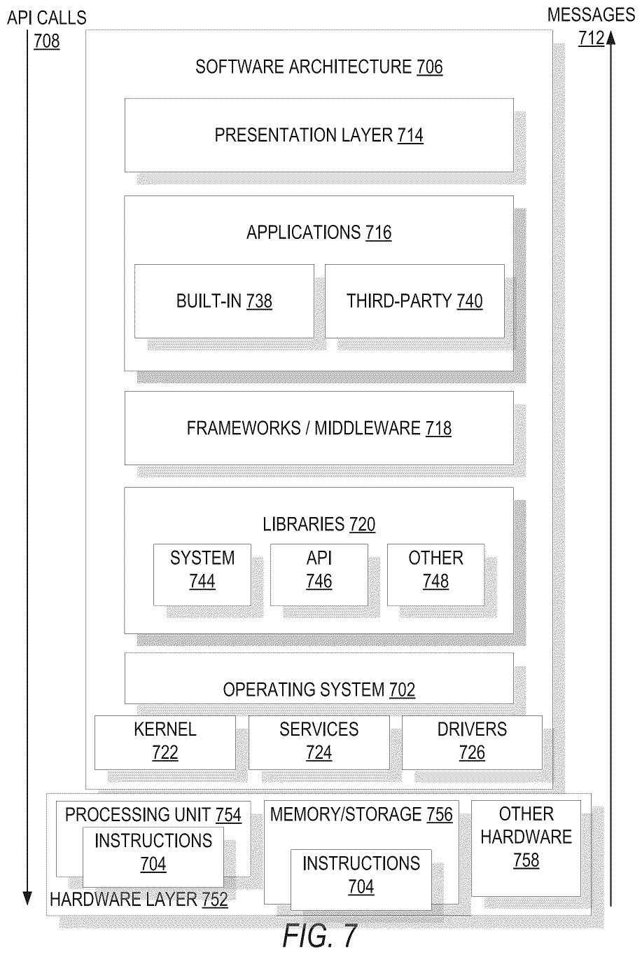

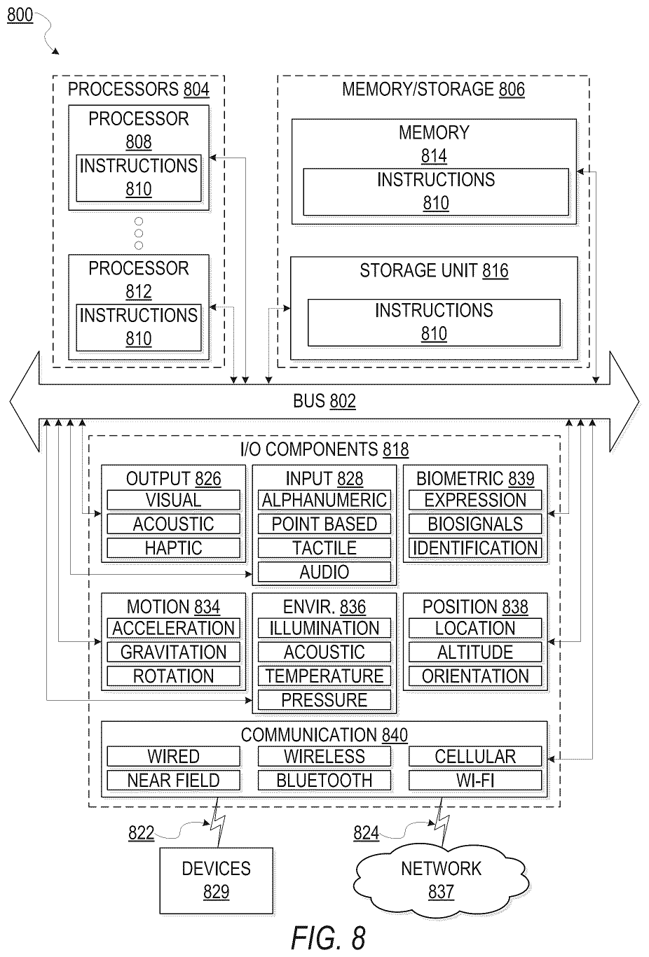

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 1. The software architecture 706 can be utilized to apply a machine learning technique or model to generate a prediction of a medical tests to recommend to a patient prior to a scheduled visit to a medical professional.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some example embodiments, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the system 100 to process a combination of patient information features with a trained machine learning model to predict or condition triggering recommendations for one or more medical tests to the patient associated with the patient information features prior to an upcoming scheduled visit to a medical professional.

As such, the instructions 810 may be used to implement devices or components described herein. The instructions 810 transform the general, non-programmed machine 800 into a particular machine 800 programmed to carry out the described and illustrated functions in the manner described. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein. The machine 800 can send and receive carrier signals that include representations of data on which the machine can operate.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, database 152, and a storage unit 816, both accessible to the processors 804 such as via the bus 802. The bus 802 can carry electrical signals, digital or analog, including carrier signals. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements, and so on. The specific I/O components 818 that are included in a particular machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting a NFC beacon signal that may indicate a particular location, and so forth.

FIG. 9 is a functional block diagram of an example neural network 902 that can be used for the inference engine or other functions (e.g., engines) as described herein to produce a predictive model. The predictive model can identify a need for one or more medical tests and can identify the one or more medical tests to recommend for particular patient information to have performed or obtained prior to an upcoming visit to a medical professional. In an example, the neural network 902 can be a LSTM neural network. In an example, the neural network 902 can be a recurrent neural network (RNN). The example neural network 902 may be used to implement the machine learning as described herein, and various implementations may use other types of machine learning networks. The neural network 902 includes an input layer 904, a hidden layer 908, and an output layer 912. The input layer 904 includes inputs 904a, 904b . . . 904n. The hidden layer 908 includes neurons 908a, 908b . . . 908n. The output layer 912 includes outputs 912a, 912b . . . 912n.

Each neuron of the hidden layer 908 receives an input from the input layer 904 and outputs a value to the corresponding output in the output layer 912. For example, the neuron 908a receives an input from the input 904a and outputs a value to the output 912a. Each neuron, other than the neuron 908a, also receives an output of a previous neuron as an input. For example, the neuron 908b receives inputs from the input 904b and the output 912a. In this way the output of each neuron is fed forward to the next neuron in the hidden layer 908. The last output 912n in the output layer 912 outputs a probability associated with the inputs 904a-904n. Although the input layer 904, the hidden layer 908, and the output layer 912 are depicted as each including three elements, each layer may contain any number of elements. Neurons can include one or more adjustable parameters, weights, rules, criteria, or the like.

In various implementations, each layer of the neural network 902 must include the same number of elements as each of the other layers of the neural network 902. For example, training patient information data features may be processed to create the inputs 904a-904n. The neural network 902 may implement a model to produce one or more medical test recommendations for at least one of the patient information data features and/or a reason for an upcoming scheduled visit to a medical professional of a particular medical professional type. More specifically, the inputs 904a-904n can include patient information data features (binary, vectors, factors or the like) stored in the storage device 110. The patient information data features can specify at least one of or combination of a reason for an upcoming visit, past medical professional recommendations, past treatment recommendations, electronic health record, past claims information for the patient, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, and/or one or more treatment preferences.

The patient information data features can be provided to neurons 908a-908n for analysis and connections between the known facts. The neurons 908a-908n, upon finding connections, provides the potential connections as outputs to the output layer 912, which determines a list of medical tests to perform or obtain from many different types of medical tests.

The neural network 902 can perform any of the above calculations. The output of the neural network 902 can be used to trigger service of care type selection to recommend to a patient in a graphical user interface. For example, the notification can be provided to a PBM, health plan manager, pharmacy, physician, caregiver, and/or a patient.

In some embodiments, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 904a is connected to each of neurons 908a, 908b . . . 908n.

The neural network 902 can use some components of the system 800 to execute the methodology as described herein.

The predictive model network 902 can further operate to map care plans to identified groups of patient data. That is, the predictive model network 902 can act to appropriately group certain patient records, which can represent one or more patients. The patient records can include data fields that may be predictive of certain care plans. The care plans can include the schedule of certain medical tests, procedures and review for a given medical state of the grouped patients. One of the care plan steps can be scheduling medical tests and/or procedures prior to an in-person visit to a medical provider, e.g., prior to an office visit with a doctor or specialist.

### Glossary

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols.

“CLIENT DEVICE” in this context refers to any machine that

interfaces to a communications network to obtain resources from one or more server systems or other client devices. A client device may be, but is not limited to, a mobile phone, desktop computer, laptop, PDA, smart phone, tablet, ultra-book, netbook, laptop, multi-processor system, microprocessor-based or programmable consumer electronics, game console, set-top box, or any other communication device that a user may use to access a network.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

“TIMESTAMP” in this context refers to a sequence of characters or encoded information identifying when a certain event occurred, for example giving date and time of day, sometimes accurate to a small fraction of a second.

Changes and modifications may be made to the disclosed embodiments without departing from the scope of the present disclosure. These and other changes or modifications are intended to be included within the scope of the present disclosure, as expressed in the following claims.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A method comprising:
receiving a request to schedule an appointment with a medical professional;
accessing patient information associated with a patient; and
in response to receiving the request to schedule the appointment, applying a predictive model, generated from historical patient records not associated with the patient, to the patient information to conditionally trigger a recommendation to obtain one or more medical tests prior to the appointment.

2. The method of claim 1, further comprising:
identifying, by the model, the one or more medical tests based on the patient information.

3. The method of claim 1, further comprising:
determining by the model that the one or more medical tests are needed prior to the appointment; and
in response to determining by the model that the one or more medical tests are needed prior to the appointment:
automatically generating a prescription to perform the one or more medical tests; and
sending the prescription to a facility associated with the patient to conduct the one or more medical tests.

4. The method of claim 1, wherein the medical professional comprises a primary care physician (PCP), and wherein the one or more medical tests comprise at least bloodwork.

5. The method of claim 1, wherein the appointment comprises an annual checkup, further comprising selecting the one or more medical tests from a plurality of available tests based on an output of the model.

6. The method of claim 1, wherein the patient information comprises at least one of an electronic health record, past claims information for the patient, patient health information, past medical recommendations, past treatment recommendations, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, or one or more treatment preferences.

7. The method of claim 1, wherein applying the model comprises:
generating a profile of patients associated with the one or more medical tests; and
determining that one or more attributes of the patient information match the profile of the patients, wherein the model is applied in response to determining that the one or more attributes of the patient information match the profile of the patients.

8. The method of claim 1, further comprising:
determining based on the patient information a date associated with a last visit the patient had with any medical professional; and
preventing recommending obtaining the one or more medical tests in response to determining that the date of the last visit is less than a threshold period of time of a current date.

9. The method of claim 1, wherein the model determines that the one or more tests are not needed prior to the appointment and prevents triggering a recommendation to perform the one or more tests prior to the appointment.

10. The method of claim 1, wherein the appointment is scheduled for a first date, wherein the one or more medical tests are prescribed to be obtained at a second date that precedes the first date, and wherein results of the one or more medical tests are made available to the medical professional prior to the first date.

11. The method of claim 1, wherein the patient information includes results of prior medical tests and diagnosis performed by one or more clinics that are different from a clinic associated with the medical professional.

12. The method of claim 1, wherein the request to schedule the appointment comprises a reason for the appointment, wherein the recommendation to obtain the one or more medical tests prior to the appointment is further generated by the model being applied to the reason for the appointment.

13. The method of claim 1, wherein the model comprises a machine learning model comprising a neural network, the machine learning model trained to establish a relationship between a plurality of training patient information features and types of medical tests performed based on visits to various types of medical professionals.

14. The method of claims 13, further comprising training the machine learning model by performing operations comprising:
obtaining a batch of training data comprising a first set of the plurality of training patient information features associated with a first type of medical test performed based on visits to a first type of medical professional;
processing the first set of the plurality of training patient information features by the machine learning model to generate an estimated set of medical tests to obtain prior to a given appointment for the first type of medical professional;
computing a loss based on a deviation between the estimated set of medical tests and the first type of medical test associated with the first set of the plurality of training patient information features; and
updating one or more parameters of the machine learning model based on the computed loss.

15. The method of claims 14, further comprising training the machine learning model by performing operations comprising:
obtaining a second batch of training data comprising a second set of the plurality of training patient information features associated with a second type of medical test performed based on visits to a second type of medical professional;
processing the second set of the plurality of training patient information features by the machine learning model to generate a second estimated set of medical tests to obtain prior to another given appointment for the second type of medical professional;
computing a second loss based on a deviation between the second estimated set of medical tests and the second type of medical test associated with the second set of the plurality of training patient information features; and
updating the one or more parameters of the machine learning model based on the computed second loss.

16. The method of claims 15, further comprising training the machine learning model by performing operations comprising:
obtaining a third batch of training data comprising a third set of the plurality of training patient information features associated with a third type of medical test performed based on visits to the second type of medical professional;
processing the third set of the plurality of training patient information features by the machine learning model to generate a third estimated set of medical tests to obtain prior to the another given appointment for the second type of medical professional;
computing a third loss based on a deviation between the third estimated set of medical tests and the third type of medical test associated with the second set of the plurality of training patient information features; and
updating the one or more parameters of the machine learning model based on the computed third loss.

17. The method of claim 13, wherein the training patient information features comprise electronic health records information and claims history.

18. The method of claim 1, further comprising:
receiving feedback from the medical professional after the appointment with the patient, the feedback indicating a level of importance of the one or more medical tests that were obtained prior to the appointment; and
retraining the model by updating one or more parameters of the model based on the triggered recommendation and the level of importance indicated in the received feedback to refine future predictions made by the model.

19. A system comprising:
one or more processors coupled to a memory comprising non-transitory computer instructions that when executed by the one or more processors perform operations comprising:
receiving a request to schedule an appointment with a medical professional;
accessing patient information associated with a patient; and
in response to receiving the request to schedule the appointment, applying a model to the patient information to conditionally trigger a recommendation to obtain one or more medical tests prior to the appointment.

20. A non-transitory computer readable medium comprising non-transitory computer-readable instructions for performing operations comprising:
receiving a request to schedule an appointment with a medical professional;
accessing patient information associated with a patient; and
in response to receiving the request to schedule the appointment, applying a model to the patient information to conditionally trigger a recommendation to obtain one or more medical tests prior to the appointment.
